(19) United States
(12) Patent Application Publication
(10) Pub. No.: US 2024/0225724 A9
(43) Pub. Date: Jul. 11, 2024

(54) POSITION TRACKING FOR PULSED FIELD ABLATION

(71) Applicant: BIOSENSE WEBSTER (ISRAEL) LTD., Yokneam (IL)

(72) Inventor: Assaf Govari, Haifa (IL)

(21) Appl. No.: 17/971,762

(22) Filed: Oct. 24, 2022

(51) Int. Cl. A61B 18/14

(52) U.S. Cl. CPC A61B 18/1492; A61B 2018/00613

(57) ABSTRACT

A system for applying a series of pulse field ablation (PFA) pulse trains to tissue of a heart includes energy-generating circuitry and a processor. The processor is configured to apply one of the pulse trains using the energy-generating circuitry and a catheter in the heart, to store a location of the catheter at which the one of the pulse trains is applied, to track a position of the catheter following the application of the one of the pulse trains, to detect, based on the tracking, that the catheter is within a predefined distance from the location following a predefined minimum duration from a most recent one of the pulse trains, and to apply another one of the pulse trains, in response to the detecting, using the energy-generating circuitry and the catheter. Other examples are also described.

### FIELD OF THE DISCLOSURE

The present disclosure is related generally to the field of electrophysiology, and particularly to the administration of pulsed field ablation (PFA).

### BACKGROUND

In PFA, an electric field causes irreversible electroporation (IRE) of the cell membrane, thereby killing the cell. Typically, a PFA routine includes the application of a series of pulse trains, with an appropriate pause between each pair of successive pulse trains.

### BRIEF DESCRIPTION OF THE DRAWINGS

The present disclosure will be more fully understood from the following detailed description of examples thereof, taken together with the drawings, in which:

FIG. 1 is a schematic illustration of an example electrophysiology mapping and ablation system, in accordance with some examples of the present disclosure; and

FIG. 2 is a flow diagram for a method for applying PFA to tissue of a heart, in accordance with some examples of the present disclosure.

### DETAILED DESCRIPTION OF EXAMPLES

### Overview

In conventional PFA routines, a series of pulse trains is applied with a fixed amount of time (e.g., 0.5-10 s) between successive trains. If the ablation catheter moves significantly, relative to the tissue, before the entire series has been applied, the series is aborted.

However, in some applications—e.g., when applying PFA within a heart of a subject—it may be difficult to avoid movement of the catheter relative to the tissue. As a result, it may be difficult to complete a PFA routine.

To address this challenge, examples of the present disclosure provide a modified PFA routine, which capitalizes on several observations:
(i) Each pulse train is typically short enough (e.g., 50-500 μs) such that any movement of the catheter during the pulse train is insignificant.
(ii) Movement of the catheter between pulse trains is often cyclical with a period of 0.5-3 s.
(iii) The pauses between successive pulse trains need not necessarily be of a fixed duration.

In view of these observations, examples of the present disclosure do not abort a PFA routine merely because the catheter moved, and do not require a fixed amount of time between successive pulses. Rather, the catheter is allowed to move cyclically between pulses. Provided the catheter returns to approximately the same location at which the most recent pulse train was applied, the next pulse train is applied.

In particular, following the application (or “delivery”) of a first pulse train at a first location, the position of the catheter is tracked. If, following a predefined minimum duration (and, typically, prior to a predefined maximum duration) from the application of the first pulse train, the catheter is at a second location within a predefined distance from the first location, a second pulse train is applied. Otherwise, the series of pulse trains is aborted.

Similarly, following the application of the second pulse train, the position of the catheter is tracked. If, following the predefined minimum duration (and, typically, prior to the predefined maximum duration) from the application of the second pulse train, the catheter is within the predefined distance from the first location and/or the second location, a third pulse train is applied. Otherwise, the series of pulse trains is aborted.

Upon aborting a series of pulse trains, a warning to the user may be outputted. Alternatively or additionally, the new location of the catheter may be selected for ablation.

### System Description

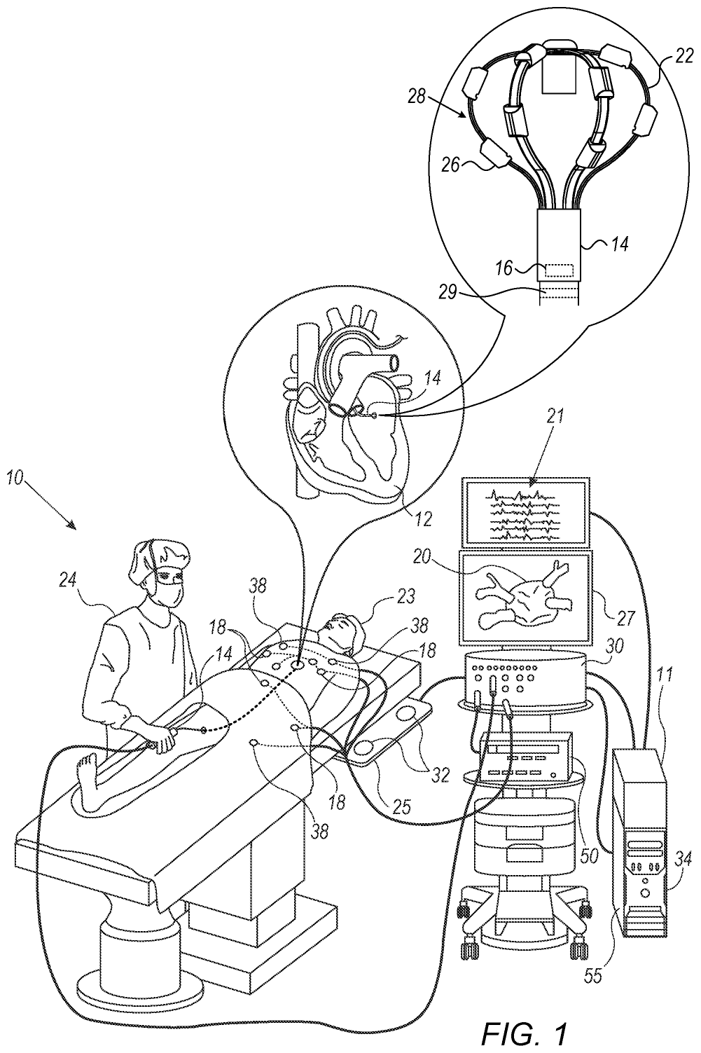

Reference is initially made to FIG. 1, which is a schematic illustration of an example electrophysiology mapping and ablation system 10, in accordance with some examples of the present disclosure. One commercial product embodying elements of system 10 is available as the CARTO™ 3 System, available from Biosense Webster, Inc., 31A Technology Drive, Irvine, CA 92618.

System 10 comprises one or more catheters, which are percutaneously inserted by a physician 24 into the body of a subject 23. Following the insertion of each catheter, physician 24 navigates the catheter through the vascular system of subject 23 into a chamber or vascular structure of the subject's heart 12. Typically, the catheters are navigated through a delivery sheath (not shown). The catheters may include a catheter for sensing intracardiac electrogram (IEGM) signals, a catheter for ablating cardiac tissue, and/or a catheter for both sensing and ablating.

For example, an exemplary catheter 14 configured for both sensing and pulsed field ablation (PFA) causing irreversible electroporation (IRE) is shown in FIG. 1. Catheter 14 comprises multiple electrodes 26 optionally distributed over a plurality of splines 22 at a distal end 28 of the catheter. Electrodes 26 are configured to sense IEGM signals from the tissue and to apply PFA pulses to the tissue, e.g., as described in co-assigned U.S. application Ser. No. 17/559,558 to Govari et al.

Typically, catheter 14 further comprises a tracking sensor 29 for tracking the three-dimensional (3D) location and orientation of distal end 28. Tracking sensor 29 may be embedded in or near distal end 28.

Typically, tracking sensor 29 comprises three magnetic coils. A location pad 25, which comprises a plurality of magnetic coils 32 configured to generate a magnetic field in a predefined working volume, is positioned near (e.g., underneath) subject 23. Based on signals induced in the coils by the magnetic field, the location and orientation of distal end 28 is tracked. Details of such magnetic-based tracking are described in U.S. Pat. Nos. 5,5391,199, 5,443,489, 5,558,091, 6,172,499, 6,239,724, 6,332,089, 6,484,118, 6,618,612, 6,690,963, 6,788,967, and 6,892,091.

Typically, system 10 further comprises one or more electrode patches 38 configured to contact the skin of subject 23. Patches 38 may establish a location reference for location pad 25. Additionally, electrical current from electrodes 26 may be sensed at patches 38, and the location of each electrode 26 may be triangulated in response thereto. This location information may be combined with the information derived from the magnetic-based tracking described above so as to increase the precision of the tracking. Details of such impedance-based tracking technology are described in U.S. Pat. Nos. 7,536,218, 7,756,576, 7,848,787, 7,869,865, and 8,456,182.

System 10 further comprises a recorder 11 and a display 27. Recorder 11 is configured to record electrocardiographic (ECG) signals 21 acquired by body-surface ECG electrodes 18 and/or IEGM signals acquired by electrodes 26 of catheter 14, and optionally, to display these signals on display 27. Recorder 11 may also be configured to pace heart 12 and/or may be electrically connected to a standalone pacer.

System 10 further comprises an ablation-energy generator 50 configured to conduct ablative energy to one or more electrodes at the distal end of an ablation catheter, such as electrodes 26. Energy produced by ablation-energy generator 50 may include, but is not limited to, radiofrequency (RF) energy and/or PFA energy, including monopolar or bipolar high-voltage direct-current (DC) pulses for effecting IRE, or combinations thereof. Ablation-energy generator 50 comprises energy-generating circuitry configured to produce the energy, along with a controller configured to control the circuitry and, optionally, perform other computational functions.

System 10 further comprises a workstation 55 comprising a processor 34, a volatile memory and/or non-volatile memory that may store appropriate software instructions, and a user interface. Processor 34 may be configured to perform multiple functions, including, for example, (1) mapping the endocardial anatomy of heart 12 in 3D and rendering the resulting anatomical map 20 for display on display 27, (2) displaying, on display 27, activation sequences and/or other data compiled from ECG signals 21 in representative visual indicia or imagery superimposed on anatomical map 20, (3) displaying the real-time location and orientation of one or more catheters within the body of subject 23, and (4) displaying sites of interest, such as sites at which ablation energy has been applied.

System 10 further comprises a patient interface unit (PIU) 30, which is configured to establish electrical communication between power supplies, workstation 55, and the electrophysiological equipment belonging to the system. The electrophysiological equipment may comprise, for example, one or more catheters, location pad 25, ECG electrodes 18, electrode patches 38, ablation-energy generator 50, and/or recorder 11. Typically, PIU 30 further comprises another processor configured to compute of location and orientation of each of the catheters and to perform any relevant computations based on ECG signals 21.

In general, the term “processor,” as used in the description and claims below, may refer to a single processor, such as processor 34, the controller of ablation-energy generator 50, or the processor of PIU 30. Alternatively, this term may refer to a cooperatively networked or clustered set of processors. For example, any of the functionality described hereinbelow may be performed cooperatively by some or all of processor 34, the controller of ablation-energy generator 50, and the processor of PIU 30.

The functionality of each of the processors described herein may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, the processor may be embodied as a programmed processor comprising, for example, a central processing unit (CPU) and/or a Graphics Processing Unit (GPU). Program code, including software programs, and/or data may be loaded for execution and processing by the CPU and/or GPU. The program code and/or data may be downloaded to the processor in electronic form, over a network, for example. Alternatively or additionally, the program code and/or data may be provided and/or stored on non-transitory tangible media, such as magnetic, optical, or electronic memory. Such program code and/or data, when provided to the processor, produce a machine or special-purpose computer, configured to perform the tasks described herein.

### Applying PFA

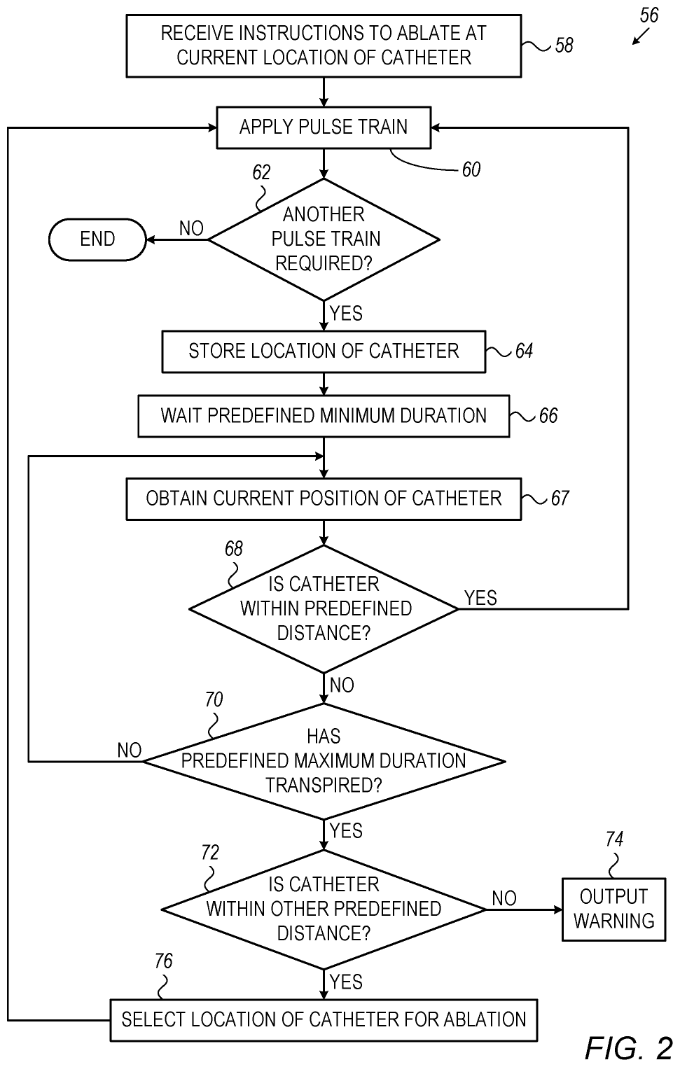

Reference is now made to FIG. 2, which is a flow diagram for a method 56, executed by a processor, for applying PFA to tissue of heart 12 using catheter 14 or any other suitable catheter, in accordance with some examples of the present disclosure.

Typically, method 56 begins with an instruction-receiving step 58, at which the processor receives instructions, from a user, to ablate tissue at the current location of the catheter. For example, subsequently to navigating the catheter to the desired location in the heart, physician 24 (FIG. 1) may enter the instructions, e.g., by pressing a foot pedal, pressing a button on the handle of the catheter, or touching a touch screen belonging to display 27. In response to the instructions, the processor begins to apply PFA to the tissue.

The processor is configured to apply PFA by applying a series of PFA pulse trains. In the context of the present application, including the claims, a “pulse train” refers to a series of one or more pulses.

The processor applies each pulse train, using the catheter, at a train-applying step 60. Subsequently to applying the pulse train, the processor checks, at a checking step 62, whether another pulse train is required, typically by comparing the number of delivered pulse trains to a predefined number. If not, method 56 ends. Otherwise, the processor stores the location of the catheter at a location-storing step 64, and then waits a predefined minimum duration, at a waiting step 66, so as not to apply the next pulse train too soon after the most recent pulse train.

Following waiting step 66, the processor tracks the position of the catheter. In other words, the processor repeatedly (i) obtains (i.e., receives or computes) the current position of the catheter at a position-obtaining step 67, and (ii) checks, at another checking step 68, whether this position is within a predefined distance from the location at which a previous pulse train was applied. (Further details regarding checking step 68 are provided below.) Thus, based on the tracking, the processor may detect that the catheter is within the predefined distance following the predefined minimum duration from the most recent pulse train. In response thereto, the processor may return to train-applying step 60.

If, on the other hand, the processor ascertains, based on the tracking, that the catheter is not within the predefined distance, the processor checks, at another checking step 70, whether a predefined maximum duration has transpired. If not, the processor returns to checking step 68. Otherwise, the processor may output a warning and/or select another location for application of PFA.

For example, the processor may check, at another checking step 72, whether the catheter is within another (larger) predefined distance from the previous location. If not, the processor may output a warning at an outputting step 74, e.g., by displaying the warning on display 27 (FIG. 1). Otherwise, the processor may, at a selecting step 76, select the location of the catheter for application of another series of PFA pulse trains. The processor may then return to train-applying step 60, optionally after receiving, from the user, approval to ablate at the new location.

Alternatively, in response to ascertaining, at checking step 70, that the predefined maximum duration has transpired, the processor may perform outputting step 74 without performing checking step 72.

In some examples, at checking step 68, the processor checks whether the catheter is within the predefined distance from the location at which the first pulse train was applied. (In such examples, location-storing step 64 may be performed only for the first pulse train of the series.) In other examples, the processor checks whether the catheter is within the predefined distance from the location at which the most recent pulse train was applied. In yet other examples, the processor checks whether the catheter is within the predefined distance from both locations, such that the next pulse train is applied only if the catheter is within the predefined distance from both locations. In yet other examples, the processor checks whether the catheter is within the predefined distance from either one of the locations, such that the next pulse train is applied provided the catheter is within either one of the locations.

In general, the predefined minimum duration, maximum duration, and distance depend on the amplitude and duration of each pulse train. In some examples, the predefined minimum duration is between 0.5 and 2 s, the predefined maximum duration is between 1 and 3 s, and/or the predefined distance is between 1 and 4 mm.

As described above, per method 56, the processor applies a subsequent pulse train upon the catheter being within the predefined distance, regardless of whether the catheter is continuing to move toward the previous location.

In other examples, the processor applies the subsequent pulse train in response to the probe reaching a minimum distance from the previous location. In other words, even after the catheter is within the predefined distance, the processor refrains from applying the next pulse train provided the catheter is continuing to move toward the previous location (and provided that the maximum duration has not yet been reached).

In some examples, processor tracks the force applied to the catheter by the tissue. For example, the processor may continually receive a force-indicating signal from a pressure transducer 16 at the distal end of the catheter. In such examples, for each of the pulse trains, the processor may apply the pulse train in response to the force exceeding a predefined lower threshold and/or being less than a predefined higher threshold. For example, prior to executing train-applying step 60, the processor may check whether the force exceeds the lower threshold and/or is less than the higher threshold. If yes, the processor may apply the pulse train; otherwise, the processor may refrain from applying the pulse train.

### EXAMPLES

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A system (10) for applying a series of pulse field ablation (PFA) pulse trains to tissue of a heart (12) includes energy-generating circuitry (50) and a processor (30, 34, 50). The processor is configured to apply one of the pulse trains, using the energy-generating circuitry and a catheter (14) in the heart. The processor is further configured to store a location of the catheter at which the one of the pulse trains is applied, to track a position of the catheter following the application of the one of the pulse trains, to detect, based on the tracking, that the catheter is within a predefined distance from the location following a predefined minimum duration from a most recent one of the pulse trains, and, in response to the detecting, to apply another one of the pulse trains using the energy-generating circuitry and the catheter.

### Example 2

The system (10) according to Example 1, wherein the processor (30, 34, 50) is configured to detect that the catheter (14) is within the predefined distance prior to a predefined maximum duration from the most recent one of the pulse trains.

### Example 3

The system (10) according to Example 2, wherein the predefined maximum duration is between 1 and 3 s.

### Example 4

The system (10) according to any one of Examples 1-3, wherein the predefined distance is between 1 and 4 mm.

### Example 5

The system (10) according to any one of Examples 1-4, wherein the predefined minimum duration is between 0.5 and 2 s.

### Example 6

The system (10) according to any one of Examples 1-5, wherein the processor (30, 34, 50) is configured to apply the other one of the pulse trains upon the catheter (14) being within the predefined distance.

### Example 7

The system (10) according to any one of Examples 1-5, wherein the processor (30, 34, 50) is configured to apply the other one of the pulse trains in response to the catheter (14) reaching a minimum distance from the location.

### Example 8

The system (10) according to any one of Examples 1-7,
wherein the series is a first series and the location is a first location, and
wherein the processor (30, 34, 50) is further configured to:
   apply a pulse train of a second series of PFA pulse trains using the energy-generating circuitry (50) and the catheter (14) in the heart (12),
   store a second location of the catheter at which the pulse train of the second series is applied,
   track the position of the catheter following the application of the pulse train of the second series,
   based on the tracking, ascertain that the catheter is not within the predefined distance from the second location following the predefined minimum duration, and prior to a predefined maximum duration, from a most recent pulse train of the second series, and
   in response to the ascertaining, output a warning.

### Example 9

The system (10) according to any one of Examples 1-7,
wherein the series is a first series and the location is a first location, and
wherein the processor (30, 34, 50) is further configured to:
   apply a pulse train of a second series of PFA pulse trains using the catheter (14) in the heart (12),
   store a second location of the catheter at which the pulse train of the second series is applied,
   track the position of the catheter following the application of the pulse train of the second series,
   based on the tracking, ascertain that the catheter is not within the predefined distance from the second location following the predefined minimum duration, and prior to a predefined maximum duration, from a most recent pulse train of the second series, and
   in response to the ascertaining, and in response to the catheter being at a third location within another predefined distance from the second location, select the third location for application of a third series of PFA pulse trains.

### Example 10

The system (10) according to any one of Examples 1-9, wherein the processor (30, 34, 50) is further configured to track a force applied to the catheter (14) by the tissue, and wherein the processor is configured to apply each of the pulse trains in response to the force exceeding a predefined threshold.

### Example 11

A method for applying a series of pulse field ablation (PFA) pulse trains to tissue of a heart (12) includes applying one of the pulse trains using a catheter (14) in the heart and storing a location of the catheter at which the one of the pulse trains is applied. The method further includes tracking a position of the catheter following the application of the one of the pulse trains and, based on the tracking, detecting that the catheter is within a predefined distance from the location following a predefined minimum duration from a most recent one of the pulse trains. The method further includes, in response to the detecting, applying another one of the pulse trains using the catheter.

### Example 12

The method according to Example 11, wherein the detecting includes detecting that the catheter (14) is within the predefined distance prior to a predefined maximum duration from the most recent one of the pulse trains.

### Example 13

The method according to Example 12, wherein the predefined maximum duration is between 1 and 3 s.

### Example 14

The method according to any one of Examples 11-13, wherein the predefined distance is between 1 and 4 mm.

### Example 15

The method according to any one of Examples 11-14, wherein the predefined minimum duration is between 0.5 and 2 s.

### Example 16

The method according to any one of Examples 11-15, wherein applying the other one of the pulse trains includes applying the other one of the pulse trains upon the catheter (14) being within the predefined distance.

### Example 17

The method according to any one of Examples 11-15, wherein applying the other one of the pulse trains includes applying the other one of the pulse trains in response to the catheter (14) reaching a minimum distance from the location.

### Example 18

The method according to any one of Examples 11-17,
wherein the series is a first series and the location is a first location, and
wherein the method further includes:
   applying a pulse train of a second series of PFA pulse trains using the catheter (14) in the heart (12);
   storing a second location of the catheter at which the pulse train of the second series is applied;
   tracking the position of the catheter following the application of the pulse train of the second series;
   based on the tracking, ascertaining that the catheter is not within the predefined distance from the second location following the predefined minimum duration, and prior to a predefined maximum duration, from a most recent pulse train of the second series; and
   in response to the ascertaining, outputting a warning.

### Example 19

The method according to any one of Examples 11-17,
wherein the series is a first series and the location is a first location, and
wherein the method further includes:
   applying a pulse train of a second series of PFA pulse trains using the catheter (14) in the heart (12);
   storing a second location of the catheter at which the pulse train of the second series is applied;
   tracking the position of the catheter following the application of the pulse train of the second series;
   based on the tracking, ascertaining that the catheter is not within the predefined distance from the second location following the predefined minimum duration, and prior to a predefined maximum duration, from a most recent pulse train of the second series; and
   in response to the ascertaining, and in response to the catheter being at a third location within another predefined distance from the second location, selecting the third location for application of a third series of PFA pulse trains.

### Example 20

The method according to any one of Examples 11-19, further including tracking a force applied to the catheter by the tissue, wherein, for each of the pulse trains, applying the pulse train includes applying the pulse train in response to the force exceeding a predefined threshold.

It will be appreciated by persons skilled in the art that the present disclosure is not limited to what has been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system for applying a series of pulse field ablation (PFA) pulse trains to tissue of a heart, the system comprising:
energy-generating circuitry; and
a processor, configured to:
using the energy-generating circuitry and a catheter in the heart, apply one of the pulse trains,
store a location of the catheter at which the one of the pulse trains is applied,
track a position of the catheter following the application of the one of the pulse trains,
based on the tracking, detect that the catheter is within a predefined distance from the location following a predefined minimum duration from a most recent one of the pulse trains, and
in response to the detecting, apply another one of the pulse trains using the energy-generating circuitry and the catheter.

2. The system according to claim 1, wherein the processor is configured to detect that the catheter is within the predefined distance prior to a predefined maximum duration from the most recent one of the pulse trains.

3. The system according to claim 2, wherein the predefined maximum duration is between 1 and 3 s.

4. The system according to claim 1, wherein the predefined distance is between 1 and 4 mm.

5. The system according to claim 1, wherein the predefined minimum duration is between 0.5 and 2 s.

6. The system according to claim 1, wherein the processor is configured to apply the other one of the pulse trains upon the catheter being within the predefined distance.

7. The system according to claim 1, wherein the processor is configured to apply the other one of the pulse trains in response to the catheter reaching a minimum distance from the location.

8. The system according to claim 1,
wherein the series is a first series and the location is a first location, and
wherein the processor is further configured to:
apply a pulse train of a second series of PFA pulse trains using the energy-generating circuitry and the catheter in the heart,
store a second location of the catheter at which the pulse train of the second series is applied,
track the position of the catheter following the application of the pulse train of the second series,
based on the tracking, ascertain that the catheter is not within the predefined distance from the second location following the predefined minimum duration, and prior to a predefined maximum duration, from a most recent pulse train of the second series, and
in response to the ascertaining, output a warning.

9. The system according to claim 1,
wherein the series is a first series and the location is a first location, and
wherein the processor is further configured to:
apply a pulse train of a second series of PFA pulse trains using the catheter in the heart,
store a second location of the catheter at which the pulse train of the second series is applied,
track the position of the catheter following the application of the pulse train of the second series,
based on the tracking, ascertain that the catheter is not within the predefined distance from the second location following the predefined minimum duration, and prior to a predefined maximum duration, from a most recent pulse train of the second series, and
in response to the ascertaining, and in response to the catheter being at a third location within another predefined distance from the second location, select the third location for application of a third series of PFA pulse trains.

10. The system according to claim 1, wherein the processor is further configured to track a force applied to the catheter by the tissue, and wherein the processor is configured to apply each of the pulse trains in response to the force exceeding a predefined threshold.

11. A method for applying a series of pulse field ablation (PFA) pulse trains to tissue of a heart, the method comprising:
applying one of the pulse trains using a catheter in the heart;
storing a location of the catheter at which the one of the pulse trains is applied;
tracking a position of the catheter following the application of the one of the pulse trains;
based on the tracking, detecting that the catheter is within a predefined distance from the location following a predefined minimum duration from a most recent one of the pulse trains; and
in response to the detecting, applying another one of the pulse trains using the catheter.

12. The method according to claim 11, wherein the detecting comprises detecting that the catheter is within the predefined distance prior to a predefined maximum duration from the most recent one of the pulse trains.

13. The method according to claim 12, wherein the predefined maximum duration is between 1 and 3 s.

14. The method according to claim 11, wherein the predefined distance is between 1 and 4 mm.

15. The method according to claim 11, wherein the predefined minimum duration is between 0.5 and 2 s.

16. The method according to claim 11, wherein applying the other one of the pulse trains comprises applying the other one of the pulse trains upon the catheter being within the predefined distance.

17. The method according to claim 11, wherein applying the other one of the pulse trains comprises applying the other one of the pulse trains in response to the catheter reaching a minimum distance from the location.

18. The method according to claim 11,
wherein the series is a first series and the location is a first location, and
wherein the method further comprises:
applying a pulse train of a second series of PFA pulse trains using the catheter in the heart;
storing a second location of the catheter at which the pulse train of the second series is applied;
tracking the position of the catheter following the application of the pulse train of the second series;
based on the tracking, ascertaining that the catheter is not within the predefined distance from the second location following the predefined minimum duration, and prior to a predefined maximum duration, from a most recent pulse train of the second series; and
in response to the ascertaining, outputting a warning.

19. The method according to claim 11,
wherein the series is a first series and the location is a first location, and
wherein the method further comprises:
applying a pulse train of a second series of PFA pulse trains using the catheter in the heart;
storing a second location of the catheter at which the pulse train of the second series is applied;
tracking the position of the catheter following the application of the pulse train of the second series;
based on the tracking, ascertaining that the catheter is not within the predefined distance from the second location following the predefined minimum duration, and prior to a predefined maximum duration, from a most recent pulse train of the second series; and
in response to the ascertaining, and in response to the catheter being at a third location within another predefined distance from the second location, selecting the third location for application of a third series of PFA pulse trains.

20. The method according to claim 11, further comprising tracking a force applied to the catheter by the tissue, wherein, for each of the pulse trains, applying the pulse train comprises applying the pulse train in response to the force exceeding a predefined threshold.
